(19) United States
(12) Patent Application Publication
(10) Pub. No.: US 2003/0108898 A1
(43) Pub. Date: Jun. 12, 2003

(54) Method for detection, quantitation and breakpoint cluster region determination of fusion transcripts

(76) Inventor: Paul Choppa, Manhattan Beach, CA (US)

Correspondence Address: GIBBONS, DEL DEO, DOLAN, GRIFFINGER & VECCHIONE, 1 RIVERFRONT PLAZA, NEWARK, NJ, 07102-5497, US

(21) Appl. No.: 10/189,292

(22) Filed: Jul. 2, 2002

Related U.S. Application Data
(60) Provisional application No. 60/302,762, filed on Jul. 3, 2001.

(51) Int. Cl. C12Q 1/68; C12P 19/34

(52) U.S. Cl. 435/6; 435/91.2

(57) ABSTRACT

A real-time RT-PCR assay is disclosed that is capable of t(15;17) fusion transcript relative quantitation and simultaneous bcr identification. This assay uses one-step chemistry, incorporates a multiplexed endogenous control and uses a novel dual probe technique to achieve in two simple reactions what has traditionally required a laborious procedure of three or more reaction followed by post-amplification analysis.

### CROSS-REFERENCE TO RELATED APPLICATIONS

This application claims priority benefits under Title 35, United States Code, 119 of provisional application of U.S. Ser. No. 60/302,762, filed Jul. 3, 2001.

### FIELD OF THE INVENTION

This invention relates generally to methods and compositions for detection and relative quantitation of specific nucleic acid sequences associated with chromosomal aberrations by forming hybrids between the sequences and genetic probes, and detecting the probes. Compositions of probes useful for detecting chromosomal translocations, in particular those associated with APL, are also disclosed.

### BACKGROUND OF THE INVENTION

Acute promyelocytic leukemia (APL) is a distinctive subtype of acute myelogenous leukemia (AML) characterized by a translocation involving the PML gene from chromosome 15 and the retinoic acid receptor α (RARα) gene from chromosome 17 [1-3]. In APL, the breakpoint on chromosome 17 is invariably located within the second intron of the RARα gene [3,4]. Fusion transcript variation exists as a result of heterogeneous breakpoint cluster regions (bcr) within the PML gene from chromosome 15 and alternative splicing of the PML sequence following transcription [5-7]. Thus, individuals with acute promyelocytic leukemia (APL) usually express one of three primary hybrid transcripts associated with a t(15;17) translocation.

The three APL fusion transcripts result from heterogeneous breakpoint cluster regions (bcr) within the PML gene and are denoted bcr1 (long), bcr2 (variant), and bcr3 (short) forms. The most 5′ breakpoint (bcr3) occurs within intron 3 of the PML gene, resulting in the fusion of PML exon 3 with RARα exon 3. The location of bcr1 is the most 3′ of the various breakpoints within the PML gene, and results in the joining of PML exon 6 and RARα exon 3. The most uncommon breakpoint, bcr2, involves inconsistent breakage within exon 6 of the PML gene resulting in the fusion of a variable portion of PML exon 6 and exon 3 of the RARα gene [8-11]. These variant cases represent ˜8-12% of t(15;17) cases. The location of bcr1, bcr2 and bcr3 results in fusion transcripts of varying lengths which are also referred to as the long (L), variant (V) and short (S) forms, respectively as described in FIG. 1.

In addition to the clinical relevance of minimal residual disease quantitation (MRD), a number of researchers have reported on the clinical relevance associated with the specific type of PML-RARα fusion transcript expressed in an individual. It has been reported that the location of the breakpoint within the PML gene may influence prognosis [11,12]. Research also indicates that identification of the specific breakpoint region may be used to predict an individual's response to all-trans retinoic acid (ATRA) treatment and the likelihood of relapse.

Although acute promyelocytic leukemia is highly curable, APL is also extremely fatal if not detected at a very early stage, regardless of the fusion transcript present. Thus, diagnosis must be made as quickly and accurately as possible to facilitate prompt treatment [20]. The standard diagnosis of APL has traditionally been morphology confirmed by cytogenetics. The microgranular variant form, in particular, usually requires cytogenetics. Microgranular variants are especially difficult to identify and may be misdiagnosed as acute myelomonocytic leukemia, resulting in inappropriate treatment [21].

All-trans retinoic acid (ATRA) provides a differentiation therapy (rather than standard cytotoxic cancer therapy) for patients with APL. Although the majority of individuals with APL respond well to treatment with ATRA, the response to treatment may depend on which form of fusion transcript is involved. Even though it has been shown to be highly effective in APL treatment, clinical resistance occurs frequently with pharmacological doses of ATRA and APL patients often relapse. Individuals expressing either the long (bcr1) or short (bcr3) form transcripts have demonstrated high sensitivity to treatment with ATRA [12-14]. Although long and short fusion transcripts initially respond well to ATRA, individuals with the short form transcript tend to have shorter periods of clinical remission than individuals expressing the long form translocation[14]. Additionally, in individuals who express the variant form, response to treatment with all-trans retinoic acid (ATRA) may depend on where the break occurs within PML exon 6 [11]. Fusion transcript sequence analysis has suggested that individuals expressing the bcr2 transcript with a breakpoint 5′ to nucleotide (nt) 1709 of the PML gene have reduced sensitivity to ATRA, while individuals expressing the variant transcript with a breakpoint 3′ to (nt) 1709 demonstrate high sensitivity to ATRA indistinguishable from individuals expressing the short or long forms [11]. Thus, there is a need to identify those patients who are likely to respond to ATRA therapy and those who are not, so that alternative therapies may be considered.

Traditionally, determination of the various fusion transcripts has been a laborious and time-consuming task [15]. Some procedures require a different reaction for each transcript, followed by electrophoresis, a hybridization reaction and, possibly, sequencing. In the case of t(15;17) translocations, improper primer placement of a variant form in traditional methods can give a false negative result if the breakpoint falls 5′ of the forward primer location.

The present invention utilizing real time PCR provides a new approach to gain insight into the relationship between heterogeneous chromosomal aberrations and their associated fusion transcripts, in particular APL and the various t(15;17) fusion transcripts involved.

### SUMMARY OF THE INVENTION

The present invention provides novel compositions and methods for simultaneously assaying multiple fusion transcripts and identifying critical breakpoints in variant transcripts in a minimal amount of time (about 4 hours), which is critical to patient care. Turn around time is minimized by using one-step chemistry and a multiplexed endogenous control. A novel multiple probe technique provides information as to the critical breakpoints in variant transcripts, eliminating the need to sequence inconclusive variant cases. It provides relative quantitative data, which may be used to monitor the course of the disease and evaluate treatment efficacy. The fact that it is probe based allows for a higher level of specificity than can be achieved by traditional gel-based methods. Assays of the present invention are capable of detecting one copy of each transcript form in a background of 100 ng of negative control RNA.

In a preferred embodiment, the present invention provides a method of evaluating t(15;17) fusion transcripts using a real-time RT-PCR capable of fusion transcript relative quantitation and simultaneous bcr identification. This assay uses one-step chemistry and incorporates a multiplexed endogenous control to normalize input RNA and serve as a determinant of the reaction success. A novel dual probe technique provides information as to the critical breakpoints in the variant cases. It has a sensitivity of 10⁻⁴.

In one aspect, the present invention provides a method of diagnosing APL in a subject which comprises detecting in a sample from the subject a nucleic acid encoding a fusion transcript associated with APL.

The present invention also provides a method of identifying a subject with a t(15;17) translocation who is more likely respond to treatment with ATRA which comprises detecting in a sample from the subject a nucleic acid encoding a bcr1 fusion transcript or a bcr3 fusion transcript or a nucleic acid containing a critical portion of the bcr2 breakpoint region sequence.

The present invention also provides a method of identifying a subject with t(15;17) translocation who is less likely to respond to treatment with ATRA which comprises detecting in a sample from the subject the presence of a nucleic acid sequence which encodes a bcr2 fusion transcript that does not contain the critical portion of the bcr2 breakpoint region.

The present invention further provides a method for monitoring the level of disease activity in a subject who has received treatment for APL which comprises monitoring the expression level of a fusion transcript associated with APL.

The present invention still further provides a method for monitoring the progress and adequacy of treatment in a subject who has received treatment for APL which comprises monitoring the expression level of a fusion transcript associated with APL at various stages of treatment.

### BRIEF DESCRIPTION OF THE DRAWINGS

FIG. 1 represents t(15;17) fusion transcript forms resulting from PML gene breakpoints at bcr1 (long form); bcr2(variant) at or 3′ to (nt)1709; bcr2(variant) at or 5′ to (nt) 1709; and bcr3 (short form);

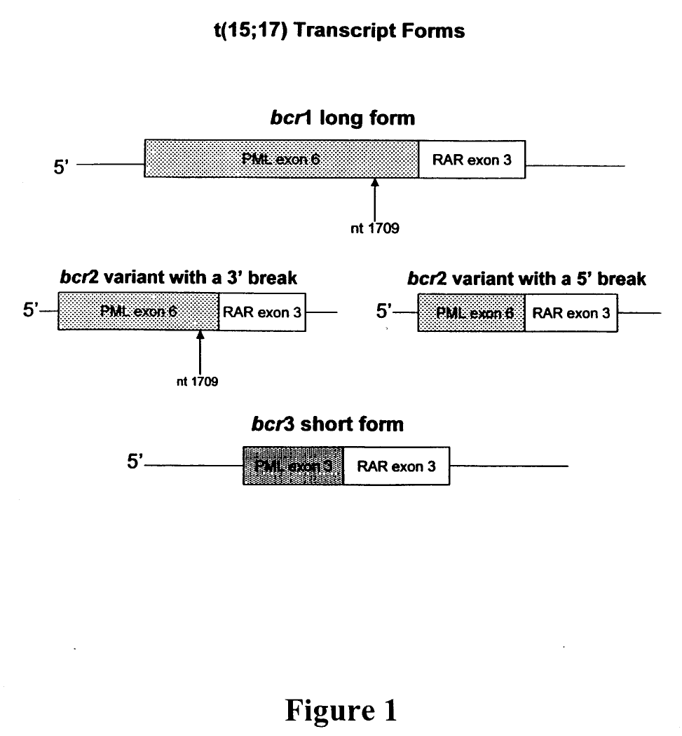

FIG. 2 represents primer and probe placement in an assay for the detection of a short form t(15;17) transcript;

FIG. 3 represents primer and probe placement in an assay for the detection of a long form t(15;17) transcript or a variant t(15;17) transcript with breakpoint 3′ to (nt) 1709 of PML exon 6 and a t(15;17) variant with breakpoint 5′ to (nt) 1709 of PML exon 6;

FIG. 4 represents primer and probe placement in an assay for detecting and specifically identifying the presence of a t(15;17) long form or a variant form with breakpoint 5′ to (nt) 1709 of PML exon 6 or a variant form with breakpoint 3′ to (nt) 1709 of PML exon 6;

FIG. 5 depicts the Amplification Plot for a t(15;17) positive sample with breakpoint 3′ to (nt) 1709 of PML exon 6. The detection of two fluorescent signals (FAM and VIC) indicates the presence of the upstream portion of PML exon 6 as well as the presence of a critical portion of the bcr2 breakpoint region including (nt)1709 of PML exon 6; and,

FIG. 6 depicts the Amplification Plot in an assay for a t(15;17) variant positive sample which has a breakpoint 5′ to (nt) 1709 of PML exon 6. The presence of the FAM signal generated from the upstream probe indicates the presence of the upstream portion of PML exon 6. No VIC signal is detected, indicating the loss of a portion of PML exon 6 5′ to (nt) 1709.

### DETAILED DESCRIPTION OF THE INVENTION

The present invention provides a method of evaluating fusion transcripts, particularly t(15;17) fusion transcripts, using a real-time RT-PCR platform that is simple and can be completed from sample to results in less time (usually about 4 hours) than traditional methods.

Real-time reverse transcriptase polymerase chain reaction methods (real-time RT-PCR) provides a platform by which many of the obstacles of traditional methods may be eliminated. Real-time RT-PCR quantitates the initial amount of the template specifically, sensitively and reproducibly, and is a preferable alternative to other forms of quantitative RT-PCR which detect the amount of final amplified product. Real-time RT-PCR monitors the fluorescence emitted during the reaction as an indicator of amplicon production during each PCR cycle (i.e., in real time) as opposed to the endpoint detection by conventional quantitative PCR methods. Real-time PCR quantitation eliminates post-PCR processing of PCR products (which is necessary in competitive RT-PCR). This helps to increase throughput, reduce the chances of carryover contamination and removes post-PCR processing as a potential source of error.

The real-time RT-PCR system is based on the detection and quantitation of a fluorescent reporter. This signal increases in direct proportion to the amount of PCR product in a reaction. By recording the amount of fluorescence emission at each cycle, it is possible to monitor the PCR reaction during the exponential phase, where the first significant increase in the amount of PCR product correlates to the initial amount of target template.

While researchers have recognized the value of real time RT-PCR in detecting the presence of fusion transcripts, the problem of primer placement has hindered the development of a real time assay for variant forms of chromosomal translocation. False negative readings could result if a forward primer or a single probe were placed in the variable region. Previous methods have avoided placing primers or probes in the variant region. This approach would not allow the accurate detection and reporting of variant forms. In the case of t(15;17) translocations, the long form expresses all of exon 6 and the vast majority of variant forms contain at least an upstream portion of exon 6. In order to avoid the variable region, prior art methods directed the probe and the forward primer to the common upstream region of exon 6, allowing the simultaneous detection of both long and variant forms in order to avoid false negative results [17].

However, in prior art methods using a single probe targeted to the common upstream region of the translocation, it is impossible to differentiate between any variant form and a long form. This differentiation is particularly critical in t(15;17) translocations involving PML exon 6, in light of indications that individuals with variant transcripts with a breakpoint at or 5′ to (nt) 1709 may exhibit a decreased sensitivity to ATRA. It is important that these individuals be identified quickly to consider alternative therapies and to avoiding losing valuable time on inappropriate therapies. This shortcoming in the prior art is overcome by the present invention assay which was designed to incorporate at least two detectable probes that target different regions of PML exon 6. Detectable labels for the probes include, but are not limited to, fluorogenic probes. Preferred fluorogenic labels include but are not limited to 6-FAM, TET and VIC. The first fluorescent probe hybridizes to an upstream portion of exon 6 common to both long and variant transcript forms; the second probe labeled with a different fluorescence hybridizes to the critical portion in the heterogenous breaking region of exon 6 which is deleted in variant transcripts with breakpoint 5′ to (nt) 1709. By placing a second probe in the critical portion of a translocation, the assay is able to detect the presence or absence of a critical breaking point of interest. Meanwhile, by directing the first probe to the common region upstream to the breakpoint, false negative results are essentially eliminated.

In embodiments employing a dual probe technique, if a long form or a variant form with a breakpoint 3′ to (nt) 1709 is present, two signals are generated, indicating the presence of the critical portion of interest containing (nt) 1709. In contrast, a variant form with a break 5′ to (nt) 1709 is readily identified by the presence of a signal corresponding to the first label only. No signal corresponding to the second label is detected in the case of a variant form with a break 5′ to (nt) 1709. The real-time assay provides relative quantitative information on the transcript expressed which may be used to monitor treatment efficacy and predict possible relapse. The novel dual probe technique is utilized to determine if the breakpoint is located 5′ or 3′ to (nt) 1709 which may be useful in evaluating an individual's response to ATRA. Thus, in this dual probe embodiment, the assay is capable of differentiating between clinically significant transcripts since individuals expressing the variant transcript with a breakpoint 3′ to (nt) 1709 demonstrate high sensitivity to ATRA indistinguishable from individuals expressing the short or long forms [11], while those individuals with a breakpoint 5′ to (nt) 1709 may demonstrate decreased sensitivity. This invention may include more than two probes used in combination with one another, each labeled with different fluorescence or other indicators if a particular translocation involves more than one critical breaking point or there are multiple regions of interest.

TaqMan probes may be used in conjunction with the present invention. The TaqMan probe is a 20-30 base long oligonucleotide that contains a reporter fluorescent dye at the 5′ end and a quencher dye at the 3′ end. The close proximity of the reporter and quencher prevents emission of any fluorescence while the probe is intact. During the reaction, cleavage of the probe separates the reporter dye and the quencher dye. This results in increased fluorescence as amplification proceeds. Accumulation of PCR products is detected by monitoring the increase in fluorescence of the reporter dye. The amount of fluorescence released during the amplification cycle is proportional to the amount of product generated in each cycle.

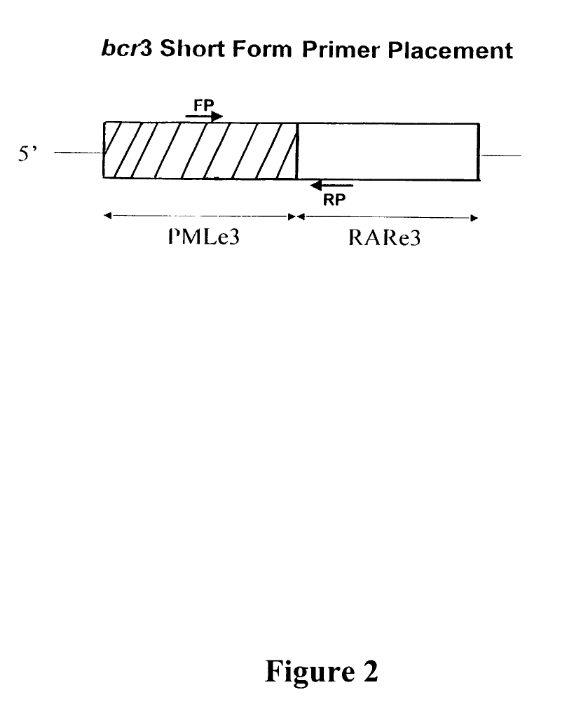

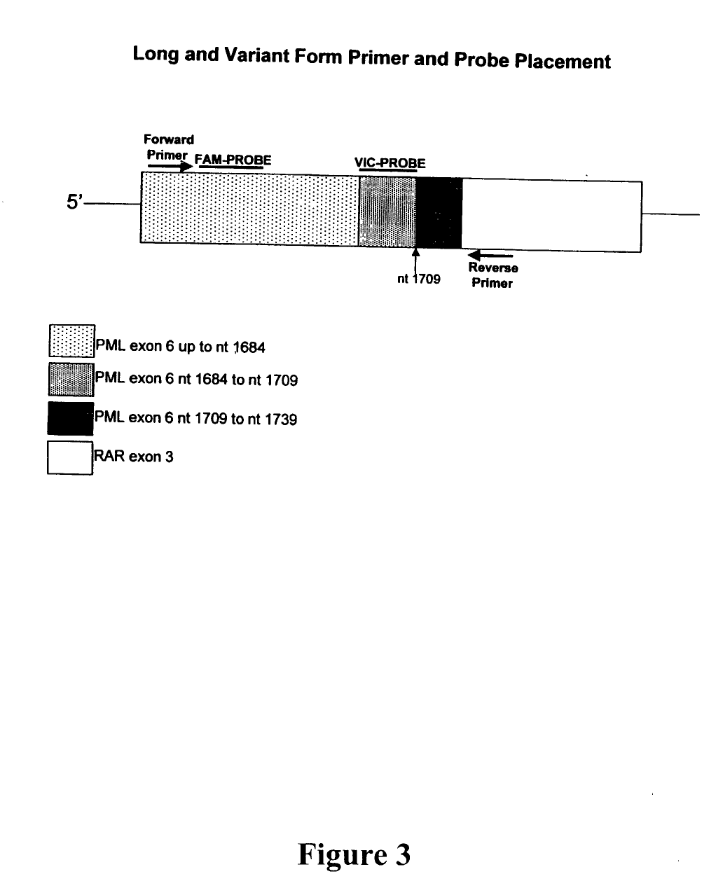

In preferred embodiments, the present invention comprises a one-step-multiplex RT-PCR. Accordingly, real time RT- PCR procedures are carried out in two separate one-step reactions, which are performed simultaneously. The first reaction amplifies and hybridizes bcr3 from a multiplex solution containing total RNA sample, bcr3 primers, a bcr3 probe, an endogenous control reference standard, reference standard primers and a reference standard probe. A preferred reference standard is β-actin. FIG. 2 illustrates primer and probe placement for bcr3 in the first reaction. The second reaction detects the bcr1 and bcr2 transcripts using a single pair of primers and two fluorogenic probes to distinguish between the long form (bcr1) and variant forms (bcr2) with a break 5′ to a predetermined critical nucleotide, specifically (nt) 1709. FIG. 3 illustrates an example of probe and primer placement in an assay for long and variant transcript forms with breakpoint 5′ to (nt)1709.A probe is placed in a critical portion of a variant breakpoint region instead of a forward primer. By utilizing a probe placed immediately 5′ to and including (nt) 1709, for example, amplification still occurs, eliminating the possibility of a false negative which would otherwise result if the breakpoint were to fall 5′ of a forward primer location. The inclusion of a second upstream probe in the same exon of the PML gene allows for the determination of a successful reaction even if the variant break falls within the sequence of the downstream probe. In this embodiment, it is not possible to differentiate between a long form and a variant form with a breakpoint 3′ to (nt) 1709. However, assuming that breakpoint 3′ to (nt) 1709 does not effect ATRA sensitivity, as research suggests, it is clinically insignificant to differentiate between these two transcript forms, both of which contain the critical portion of the break point region which includes (nt) 1709 [11].

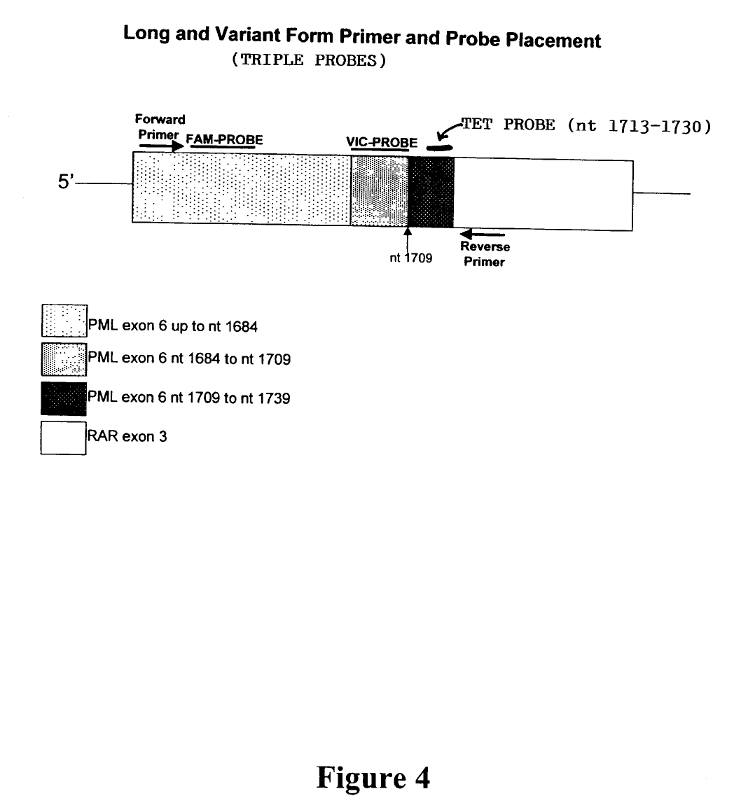

By utilizing a third probe or a probe with a third detectable label, the assay is capable of differentiating between a long form t(15,17) translocation and a variant form with breakpoint 3′ to (nt). As shown in FIG. 4, the primers and the first and second probes may be placed as described above. The third probe is directed sufficiently 3′ to (nt) 1709 to target the region of exon 6 that is missing in both variant forms but is present in the long form. Since the long form contains all of exon 6, the third probe may be placed at the extreme 3′ end of exon 6, a region which is not common to either variant. Alternatively, the third detectable probe is targeted to bridge the extreme 3′ end of exon 6 and the adjacent 5′ end of RARα portion of the transcript.

Quantitation of the involved transcript may be determined in relation to a positive control series which is used in this assay and normalized to β-actin expression. This assay uses the standard curve method for multiplexed samples in a single tube to determine relative quantitative values. The assay method of the present invention is sensitive, rapid, and minimizes labor. It provides accurate relative quantitation of all three fusion transcripts and is also capable of determining the specific breakpoint involved which may be used by clinicians to determine treatment and likelihood of relapse.

In summary, the present invention provides the use of real time RT-PCR as a platform in assaying chromosomal translocations which include heterogenous breakpoints, such as t(15;17) in acute promyelocytic leukemia. It eliminates many of the obstacles of traditional methods. For example, by placing a probe in the critical breakpoint region of a variant instead of the forward primer, amplification will still occur and the possibility of a false negative result is eliminated. As shown in the Examples that follow, the inclusion of a FAM labeled probe in the upstream region of PML exon 6 allows for the determination of a successful reaction even if the variant break falls within the sequence of the downstream VIC labeled probe. This one-step multiplex real time RT-PCR is sensitive, specific and can identify the long, short, and clinically relevant variant transcript forms associated with APL. It provides relevant quantitative information about the involved transcript. Additionally, it provides more information about the t(15;17) translocation in substantially less time than traditional methodologies.

The identification of specific fusion transcripts is clinically relevant in a number of other diseases that are characterized by chromosomal aberrations. For example, the Philadelphia (Ph.¹) chromosome, is found in chronic myelogenous leukemia (CML) and acute lymphoblastic leukemia (ALL) associated with the BCR-ABL transcript. The Ph.¹ chromosome is associated with a high relapse rate and short survival. BCR-ABL transcripts are the result of a translocation of the proto-oncongene ABL (chromosome 9) to the breakpoint cluster region (BCR) (chromosome 22). The t(9;22) translocation leads to two major types of BCR-ABL fusion genes. One major type has a BCR breakpoint in the limited region of the M-bcr and produces a 210 kd protein. This is the type of fusion gene found in virtually all cases of CML. The other major type of fusion gene has a BCR breakpoint in the large region of the BCR first intron and it produces a 190 kd protein. This type of fusion gene is associated with Ph¹ positive cases of ALL, carrying a poor prognosis. The accurate detection of Ph.1 is thus an important part of the diagnostic evaluation of patients with ALL. In addition, the t(4;11) t(9;11) and t(11;19) chromosome translocations on the ALL-1 gene on chromosome 11 each result in two reciprocal fusion products coding for different chimeric proteins. Identifying the breakpoint cluster region and the genes involved in these chromosome 11 aberrations associated with acute leukemias would be a valuable tool in providing diagnostics and therapeutics for these diseases.

The following examples further illustrate but do not limit the present invention.

### EXAMPLE 1

### Specimens and Controls

Specimens—Peripheral blood and bone marrow samples were collected from individuals who were determined to have a t(15; 17) translocation by standard RT-PCR procedures as well as negative control samples. As part of the validation process, all positive samples used in this assay were also tested by FISH, cytogenetics or traditional RT-PCR by an independent laboratory. Sequencing was performed on the samples positive for the variant transcript to determine the exact location of the breakpoint involved.

Controls—The NB4 cell line was used as a positive control for the bcr1 long form [16]. The t(15;17) translocation which is characteristic of APL is stably carried in NB4 cells and the PML/RARα fusion protein is expressed in the transformed cell line. The positive control for the bcr3 reaction was obtained from patient RNA expressing the short form fusion transcript. All controls were diluted over a range of 1000 copies to 1 copy of the fusion transcript from an RNA stock solution of 10 ng/μl. The diluted controls were brought to a final RNA concentration of 10 ng/μl using RNA extracted from the negative control cell line (HL-60). All positive samples used to validate this assay were tested by cytogenetics or traditional RT-PCR and sequencing if necessary to determine the fusion transcript form and exact location of the breakpoint for variant messages.

### EXAMPLE 2

### RNA Extraction

Total RNA was isolated from 300 μl of whole blood or bone marrow using the Qiagen RNA Mini Blood Kit followed by DNase treatment directly on the RNA binding column using the Qiagen RNase-free DNase kit (Chatsworth, Calif.). The RNA was quantified using a Molecular Devices SpectraMax Plus spectrophotometer. All RNA samples were brought to a final concentration of 10 ng/μl. Each sample was determined to be free of contaminating genomic DNA using a standard β-actin (-RT) PCR prior to RT-PCR analysis (data not shown). Ten μl (100 ng) of each sample were used in a one-step RT-PCR reaction using the EZ One-step TaqMan RT-PCR kit (Perkin-Elmer, Foster City, Calif.).

### EXAMPLE 3

### Primers and Probes

The primers and probes for this assay were designed using Primer Express 1.0 (Perkin Elmer/Applied Biosystems, Foster City, Calif.) as follows:

(SEQ. ID NO.1)

bcr1 and bcr2 forward primer (FP FIG. 3):
5′-TCT CCA ATA CAA CGA CAG CCC-3′

(SEQ. ID NO.2)

bcr1 probe (probe 2 - FIG. 3):
5′6-FAM- AGG AAG TGC AGC CAG ACC CAG TGC-3′

(SEQ. ID NO.3)

bcr2 probe (probe 3 - FIG 3):
5′VIC AAG TGA GGT CTT CCT GCC CAA CAG CA-3′

(SEQ. ID NO.10)

bcr2 long probe (probe 4, FIG. 4)
5′VIC ACG TGG CCA GTG GCG CCG-3′

(SEQ. ID NO.4)

bcr3 forward primer (FP FIG. 2):
5′GAT GGC TTC GAC GAG TTC AAG-3′

(SEQ. ID NO.5)

bcr3 probe (probe 1 FIG. 2)
5′6FAM-AGG ACC TCA GCT CTT GCA TCA CCC AG-3′

(SEQ. ID NO.6)

bcr1, bcr2, bcr3 reverse primer (RP FIGS. 2 and 3)
5′-GCA CTA TCT CTT CAG AAC TGC TGC-3′

(SEQ. ID NO.7)

β-actin forward primer:
5′TCA CCC ACA CTG TGC CCA TCT ACG A-3′

(SEQ. ID NO.8)

β-actin reverse primer:
5′CAG CGG AAC CGC TCA TTG CCA ATG G-3′

(SEQ. ID NO.9)

β-actin probe:
5′VIC-ATG CCC TCC CCC ATG CCA TCC TGC GT-3′

### EXAMPLE 4

### One-Step Real-Time RT-PCR

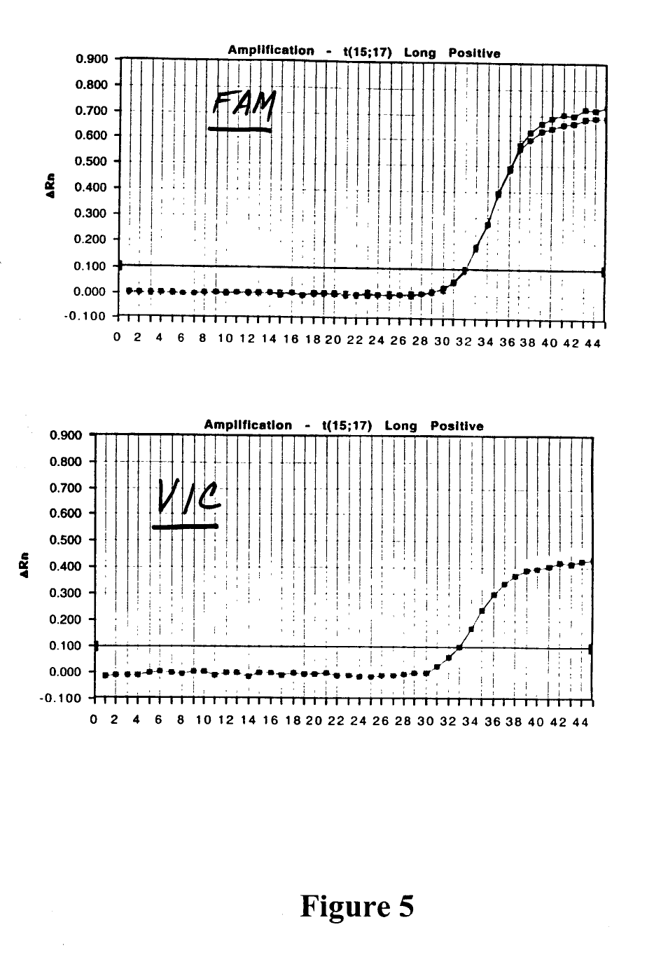

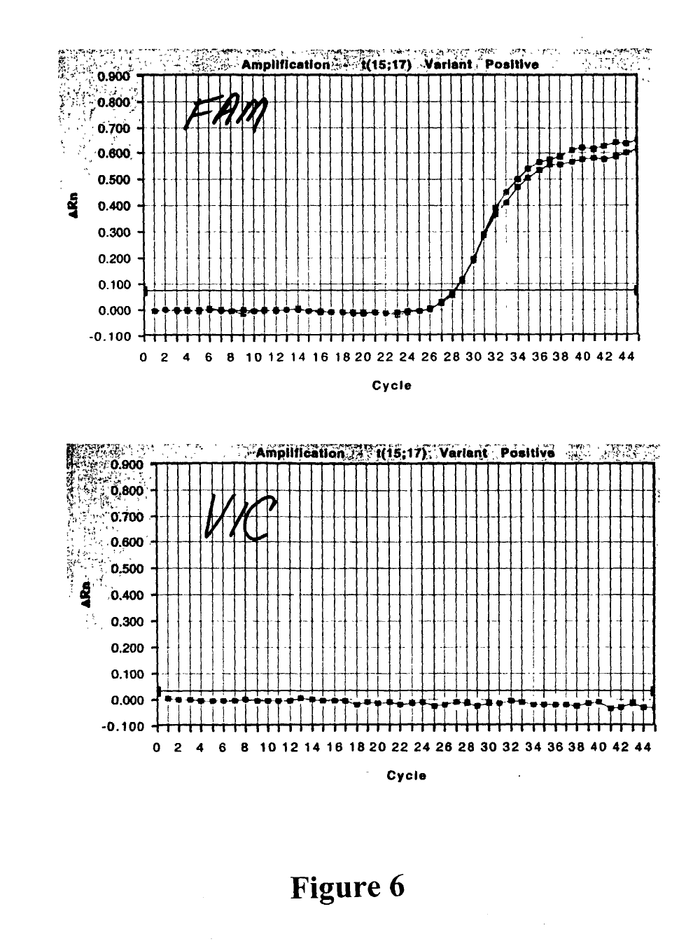

This procedure was performed in two separate reactions, but was set up and performed at the same time. The first reaction multiplexed the bcr3 primers and 6-FAM probe with the β-actin primers and VIC probe. FIG. 2 illustrates primer and probe placement in the first reaction. The second reaction detected the bcr1 and bcr2 transcripts using a single pair of primers and two fluorogenic probes. A probe was placed in the critical region of a variant breakpoint instead of a forward primer. Thus it was possible to specifically identify a variant form with break 5′ to (nt) 1709 (FIG. 6). With this probe placement, it was not possible to differentiate between a long form and a variant form with a breakpoint 3′ to (nt) 1709 (FIG. 5), however, this is probably clinically insignificant in testing for ATRA sensitivity, since research has suggested that a break 3′ to (nt)1709 does not effect ATRA sensitivity.

The reaction conditions were the same for both the bcr3/β-actin and the bcr1/bcr2 reactions. Each reaction was performed in a 25 μl reaction volume consisting of 3 mM Mn(Oac)₂, 1× EZ One-step RT-PCR buffer, 0.4 μM dNTP, 0.1 μM of all PML-RARα primers and probes, 0.025 μM β-actin primers and probe, 0.25 U Uracil N-glycosylase, and 2.5 U rTth polymerase. The reaction mixtures were placed in an ABI PRISM 7700 Sequence Detector and subjected to the following conditions: Hold 2 min at 50.0° C. (Contamination control); Hold 30 min at 60.0° C. (Reverse Transcription); followed by 45 cycles of 15 seconds at 95.0° C. (Denaturation) and 1 min at 60.0° C. (Anneal/Elongation). The ABI PRISM 7700 uses a charged coupled device (CCD) camera to collect emission data generated from the cleavage of a sequence specific fluorogenic probe during PCR. The data is then analyzed by the Sequence Detection System (SDS) software and displayed as an amplification plot of relative fluorescence vs. cycle number.

### EXAMPLE 5

### Results

The RT-PCR procedure was evaluated for sensitivity, specificity, linearity and correlation with traditional t(15;17) detection methodologies. The assay was then used to characterize the translocation forms in 25 acute promyelocytic leukemia (APL) cases. The sensitivity of the present invention assay was determined by diluting positive control total RNA from the NB4 cell line into negative control RNA. Each positive control was brought to a final concentration of 10 ng/μl with negative control RNA to maintain consistent quantities of background RNA throughout the dilution series. The positive controls which consisted of 1000, 100, 10, 5, 2 and 1 copy/10 μl maintained reproducible linearity between assays. These dilutions were based on a recent report that determined through real-time quantitative RT-PCR that there are ˜10⁵ copies of the long form fusion transcript per 1 ug of total RNA contained in the NB4 cell line [18]. Based on this report, the dilution series used roughly represents a range of 1000 copies to less than 1 copy of the t(15;17) transcript/10 ul input RNA. The assay consistently detected the 100 pg control in both reactions, equivalent to the detection of 1 single copy of controls in both reactions. The positive control RNA for the bcr3 form was derived from a positive patient sample expressing a t(15; 17) short form transcript. The method was completed from sample to results in about 4 hours. The bcr3/β-actin and bcr1/bcr2 reactions were both capable of maintaining reproducible linearity over the dilution range with slopes ranging from −3.3 to −3.4 and an r fit of >0.99.

This procedure had a correlation of 100% when compared to traditional methods for detection of t(15;17) fusion transcripts and identification of bcr2 breakpoints associated with a possible reduced sensitivity to ATRA. Each sample was evaluated by one or more validated methodologies to confirm a t(15;17) translocation. The confirmatory procedures included fluorescence in situ hybridization (FISH), cytogenetics, RT-PCR, and sequencing. This assay had a correlation of 100% when compared to traditional methods for detection of the three fusion transcripts associated with a t(15;17) translocation, and identification of bcr2 breakpoints 5′ to (nt) 1709.

Quantitation of the involved transcript was determined in relation to the positive control series in this assay and normalized to β-actin expression. The assay uses the standard curve method for multiplexed samples in a single tube to determine relative quantitative values [19].

A total of 25 positive t(15;17) samples were evaluated. The assay successfully identified the different forms of t(15;17) translocation. Twelve (48%) were identified as expressing the long form transcript, ten (40%) expressed the short form and three (12%) expressed the variant transcript. The three variant samples were comprised of 2 cases with a breakpoint 5′ to (nt) 1709 and 1 with a breakpoint 3′ to (nt) 1709. Fusion transcript sequence analysis of the three variant samples was performed to determine the exact breakpoint involved. The two variant cases with a break 5′ to (nt) 1709 both contained breakpoints at (nt) 1685. This supports previous findings which identify (nt) 1685 as the most common breakpoint found in variant samples. The variant case with a break 3′ to (nt) 1709 contained a breakpoint at (nt) 1718. Additional assays were performed on all cases positive for a break 3′ to 1709 with a probe targeting the 3′ region of 1709 and the variant case with 3′ break was successfully identified. As discussed herein, the addition of a third labeled probe targeting the 3′ region of 1709 allows the present invention to simultaneously determine the breakpoints within variants.

Amplification plots generated from patient samples expressing the long form fusion transcript demonstrated consistent Ct values between the upstream and downstream fluorogenic probes indicating the presence of an intact PML exon 6. In the variant sample which had a breakpoint at or 3′ to (nt) 1709, the signal was indistinguishable from that of a signal generated from a long form. (FIG. 5). The variant samples with a break 5′ to (nt) 1709 showed a complete loss of the VIC signal demonstrating the loss of a portion of PML exon 6, 5′ to (nt) 1709 (FIG. 6).

When the procedure above was applied in alternative embodiments utilizing a TET fluorogenic marker on the bcr2 probe (SEQ. ID NO.3), variant samples which had a breakpoint at or 3′ to (nt) 1709, the signal was indistinguishable from that of a signal generated from a long form. A variant sample with a break 5′ to (nt) 1709 showed a dramatic change in the TET signal if not a complete loss. A variant with a breakpoint at (nt) 1702 generated a weak signal from the TET probe as enough homology remained between the PML sequence and probe to cause some hybridization of the probe to its target (data not shown). The weak signal generated from the TET labeled probe may be eliminated by using a different fluorochrome.

A major advantage of this system is that it allows for detection of the various transcripts and gives information as to the critical breakpoints in the variant cases in a single run. It provides significant information with a very rapid turn around time and eliminates the need to sequence variant cases to determine if the break falls 5′ or 3′ to (nt) 1709 of PML exon 6.

It is understood that various other embodiments and modifications in the practice of the invention will be apparent to, and can be readily made by, those skilled in the art without departing from the scope of the invention described above. Accordingly, it is not intended that the scope of the claims appended hereto be limited to the exact description set forth above, but rather that the claims be construed as encompassing all of the features of patentable novelty which reside in the present invention, including all the features and embodiments which would be treated as equivalents thereof by those skilled in the art to which the invention pertains.

### References

The references listed below are incorporated herein by reference to the extent that they supplement, explain, provide a background for, or teach methodology, techniques, and/or compositions employed herein.

1. de The H, Chomienne C, Lanotte M, Degos L, Dejean A: The t(15;17) translocation of acute promyelocytic leukemia fuses the retinoic acid receptor a gene to a novel transcribed locus. Nature 1990;347:558 561w

2. Kakizuka K A, Miller W H Jr., Umesono K. et al: Chromosomal translocation t(15;17) in human acute promyelocytic leukemia fuses RARα with a novel putative transcriptional factor, PML. Cell 1991;66:663 674

3. de The H, Lavau C, Marchio A, Chomienne C, Degos L, Dejean A: The PML-RARα fusion mRNA generated by the t(15;17) translocation in acute promyelocytic leukemia encodes a functionally altered RAR. Cell 1991;66:675 684

4. Borrow J, Goddard A D, Sheer D, Solomon E: Molecular analysis of acute promyelocytic leukemia breakpoint cluster region on chromosome 17. Science 1990;249:1577 1580

5. Pandolfi P P, Alcalay M, Fagioli M, et al: Genomic variability and alternative splicing generate multiple PM/RAR alpha transcripts that encode aberrant PML proteins and PMURAR alpha isoforms in acute promyelocytic leukemia. EMBO J 1992;11:1397 1407

6. Dong S, Geng J P, Tong J H, et al: Breakpoint clusters of the PML gene in acute promyelocytic leukemia: Primary structure of the reciprocal products of the PMURARα gene inpatients with t(15;17). Genes Chromosomes Cancer 1993;6:133 139

7. Tong J H, Dong S, Geng J P, et al: Molecular rearrangements of the myl gene in acute promyelocytic leukemia (APL M3) define a breakpoint cluster region as well as some molecular variants. Oncogene 1992;7:311 316

8. 8. Chen S J, Chen Z, Chen A, et al: Occurrence of distinct PML RARα fusion gene isoforms in patients with acute promyelocytic leukemia detected by reverse transcriptase polymerase chain reaction. Oncogene 1992;7:1223 1232

9. 9. Miller W H Jr., Kakizuka A, Frankel S R, et al: Reverse transcription polymerase chain reaction for the rearranged retinoic acid receptor a clarifies diagnosis and detects minimal residual disease in acute promyelocytic leukemia. Proc Natl Acad Sci 1992;89:2694 2698

10. 10. Fukutani H, Naoe T, Yoshida H, Yamamori S, Ohno R: Molecular heterogeneity of the PML gene rearrangement in acute promyelocytic leukemia: Prevalence and clinical significance. Jpn J Cancer Res 1993;84:257 264

11. 11. Gallagher R E, Li Y P, Rao S, et al: Characterization of acute promyelocytic leukemia cases with PML RARα break/fusion sites in PML exon 6: Identification of a sub group with decreased in vitro responsiveness to all trans retinoic acid. Blood 1995;86:15401547

12. 12. Huang W, Sun G L, Li Z s, et al: Acute promyelocytic leukemia: Clinical relevance of two major PML RARα isoforms and detection of minimal residual disease by reverse transcriptase polymerase chain reaction to predict relapse. Blood 1993;82:1264 1269

13. 13. Vahdat L, Maslak P, Miller W H Jr., et al: Early mortality and retinoic acid syndrome in acute promyelocytic leukemia: Impact of leukocytosis, low dose chemotherapy, PMLRARα isoforms and CID 13 expression in patients treated with all trans retinoic acid. Blood 1994;84:3843 3849

14. Jurcic J G, Nimer S D, Scheinberg D A, DeBlasio T, Warrell R P Jr., Miller W H Jr: Prognostic significance of minimal residual disease detection and PML RARα isoform type: Long term follow up in acute promyelocytic leukemia. Blood 2001;98:2651 2656

15. Hussey C E, Lyon E, Millson A, Lay M J, Wittwer C T, Segal G H: A rapid practical RT-PCR based approach for the detection of the PML/RAR alpha fusion transcript in acute promyelocytic leukemia. Am J Clin Pathol 1999;112:256 262

16. Lanotte M, Martin Thouvenin V, Najman S, Ballerini P, Valensi F, Berger R: A maturation inducible cell line with t(15;17) marker isolated from a human acute promyelocytic leukemia (M3). Blood 1991;77:1080 1086

17. Slack J L, Williams C L, Waderson J, et al: Molecular analysis and clinical outcome of adult APL patients with the type V PML RARα isoform: Results from intergroup protocol 0129. Blood 2000;95:398 403

18. Cassinat B, Zassadowski F, Balitrand N, et al: Quantitation of minimal residual disease in acute promyelocytic leukemia patients with t(15;17) translocation using real time RT-PCR. Leukemia 2000;14:324 328

19. PE Applied Biosystems: Relative Quantitation of Gene Expression. User Bulletin #2 1997:16 24

20. Oconnor S, Evans P, Morgan G J: Diagnostic approaches to acute promyelocytic leukaemia. Leukemia and Lymphoma 1999;33:53 63

21. Foucar K: Bone Marrow Pathology, 2nd ed. ASCP Press, Chicago, 2001

What is claimed is:

1. A method of diagnosing promyelocytic leukemia resulting from a t(15;17) PML/RARα gene translocation comprising:
assaying for, in a nucleic acid sample from a subject, a nucleic acid of bcr3, a bcr nucleic acid containing a critical portion of the breakpoint region associated with bcr2, or a bcr2 nucleic acid not containing said critical portion, wherein the presence of any of these nucleic acids is indicative of promyelocytic leukemia associated with bcr1, bcr2 with breakpoint 3′ to said critical portion, bcr2 with breakpoint 5′ to said critical portion, or bcr3.

2. The method of claim 1 wherein said critical portion includes nucleotide 1709 of PML exon 6

3. A method of identifying a subject with acute promyelocytic leukemia resulting from a t(15;17) PML/RARα gene translocation who will respond to treatment with all-trans retinoic acid comprising the steps of
(a) obtaining a sample which contains sample nucleic acid from a subject
(b) contacting a first portion of sample nucleic acid from step (a) in a first reaction with a pair of bcr3 primers and a bcr3 probe specific for a bcr3 nucleic acid sequence under one-step real time RT-PCR reaction conditions suitable for nucleic acid amplification and hybridization of complementary nucleic acid sequences wherein an amplification product is formed if the sample contains a bcr3 nucleic acid;
(c) contacting a second portion of sample nucleic acid from step (a) in a second reaction with a primer pair specific for a bcr1 nucleic acid sequence, and a pair of probes with different detectable labels comprising an upstream probe and a downstream probe, wherein the upstream probe is specific for a bcr1 nucleic acid located upstream in the same exon as a predetermined bcr2 breakpoint region and the downstream probe is specific for a nucleic acid that sequences a critical portion of said bcr2 breakpoint region, said second reaction being carried out under one-step real time RT-PCR reaction conditions suitable for nucleic acid amplification and hybridization of complementary nucleic acid sequences, wherein an amplification product is formed if the sample contains a bcr1 nucleic acid or a bcr2 nucleic acid; and,
d) determining whether an amplification product is present as a result of said first reaction or said second reaction by detecting said probes, such that the detectable presence of a bcr3 amplification product or an amplification product containing the critical portion of the breakpoint region associated with bcr2 indicates that the subject will respond to treatment with all-trans retinoic acid.

4. The method of claim 3 wherein said first reaction and said second reaction are carried out simultaneously.

5. The method of claim 3 wherein said first reaction is a multiplex reaction including an endogenous control reference nucleic acid, primers specific for said reference nucleic acid and at least one probe specific for said reference nucleic acid.

6. The method of claim 3 wherein said critical breakpoint region comprises a nucleic acid sequence including nucleotide 1709 PML exon.

7. A pair of oligonucleotide primers for the amplification of bcr1 or bcr2 nucleic acid but not bcr3 nucleic acid, comprising oligonucleotides containing the following nucleic acid sequences:
(SEQ. ID NO.1)
5′-TCT CCA ATA CAA CGA CAG CCC-3′
(SEQ. ID NO.6)
5′-GCA CTA TCT CTT CAG AAC TGC TGC-3′.

8. A pair of oligonucleotide primers for the amplification of bcr3 nucleic acid, but not bcr1 or bcr2 nucleic acid, comprising oligonucleotides containing the following nucleic acid sequences:
(SEQ. ID NO.4)
5′-GAT GGC TTC GAC GAG TTC AAG-3′
(SEQ. ID NO.6)
5′-GCA CTA TCT CTT GAG AAC TGC TGC-3′.

9. A probe comprising the nucleotide sequence 5′ AGG AAG TGC AGC CAG ACC CAG TGC-3′ (SEQ. ID NO. 2) that hybridizes to an amplified target nucleic acid sequence resulting from the use of the primer pair of claim 7 in a polymerase chain reaction said target nucleic acid sequence located sufficiently 5′ to (nt) (1709) to be present in bcr1, bcr2 with breakpoint 3′ to (nt) 1709 and bcr2 with breakpoint 5′ to (nt) 1709 of PML exon 6.

10. A probe comprising the nucleotide sequence 5′AAG TGA GGT CTT CCT GCC CAA CAG CA-3′ (SEQ. ID NO. 3) that hybridizes to an amplified target nucleic acid sequence resulting from the use of the primer pair of claim 7 in a polymerase chain reaction, said target nucleic acid sequence including (nt) 1709.

11. A probe comprising the nucleotide sequence 5′-AGG ACC TCA GCT CTT GCA TCA CCC AG-3′(SEQ. ID NO. 5) that hybridizes to an amplified target nucleic acid sequence resulting from the use of the primer pair of claim 8 in a polymerase chain reaction.

12. A kit for use in detecting the presence of a t(15;17) PML/RARα gene translocation in a sample suspected of containing said translocation comprising:
(a) a first container;
(b) a primer pair of claim 7 in said first container
(c) a second container; and,
(d) a primer pair of claim 8 in said second container.

13. The kit of claim 12 further comprising:
A first probe comprising the nucleotide sequence 5′-AGG ACC TCA GCT CTT GCA TCA CCC AG-3′ (SEQ. ID NO. 5) that hybridizes to an amplified target nucleic acid sequence resulting from the use of the primer pair of claim 8 in a polymerase chain reaction.
A second probe comprising the nucleotide sequence 5′ AGG AAG TGC AGC CAG ACC CAG TGC-3′ (SEQ. ID NO. 2) that hybridizes to an amplified target nucleic acid sequence resulting from the use of the primer pair of claim 7 in a polymerase chain reaction said target nucleic acid sequence common to bcr1, bcr2 with breakpoint 3′ to (nt) 1709 and bcr2 with breakpoint 5′ to (nt) 1709.
A third probe comprising the nucleotide sequence 5′ AAG TGA GGT CTT CCT GCC CAA CAG CA-3′ (SEQ. ID NO. 3) that hybridizes to an amplified target nucleic acid sequence resulting from the use of the primer pair of claim 7 in a polymerase chain reaction, said target nucleic acid sequence common to bcr1 and bcr2 with breakpoint 3′ to (nt) 1709 and located at a critical breakpoint region 3′ to said second probe and immediately 5′ to (nt) 1709.

14. The kit of claim 13 further comprising means for detecting the hybridization of said probes to said amplified target nucleic acid sequences.

15. The kit of claim 13 further comprising
A fourth probe comprising the nucleotide sequence 5′ ACG TGG CCA GTG GCG CCG-3′ (SEQ. ID NO.10) that hybridizes to an amplified target nucleic acid sequence resulting from the use of the primer pair of claim 7 in a polymerase chain reaction, said target nucleic acid sequence present in bcr1 and not present in bcr2.

16. A kit for use in detecting the presence of target nucleic acid translocation sequences and further detecting heterogeneous breakpoints in said translocation sequences in a sample suspected of containing said translocation, said kit comprising:
a) A container
b) a pair of oligonucleotide primers in said container, said primer pair capable of amplifying a plurality of target translocation sequences, said target translocation sequences having common nucleic acid sequences flanking a heterogeneous breakpoint region;
c) An upstream probe comprising a nucleotide sequence that hybridizes to an amplified upstream target nucleic acid sequence resulting from the use of said primer pair in a polymerase chain reaction, said upstream target nucleic acid sequence located in the same exon as a critical breakpoint region in said heterogeneous breakpoint region.
d) A downstream probe comprising a nucleotide sequence that hybridizes to an amplified critical target nucleic acid resulting from the use of said primer pair in said polymerase chain reaction, said critical target nucleic acid located in a critical breakpoint region, said critical nucleic acid not present in at least one of said plurality of translocation sequences.

17. A method for determining the presence of a clinically significant target fusion transcript in a subject suspected of having said fusion transcript comprising the steps of
(a) obtaining a sample which contains sample nucleic acid from a subject;
(b) contacting a first portion of sample nucleic acid from step (a) in a first reaction with a primer pair specific for a common target nucleic acid sequence specifically associated with a first, a second and a third target fusion transcript and a pair of probes comprising an upstream probe and a downstream probe with different detectable labels, wherein said upstream probe is specific for a first target nucleic acid associated specifically with all of said first, second and third target fusion transcripts and the downstream probe is specific for a second nucleic acid comprising a critical portion of a heterogeneous breakpoint region, said critical portion specifically associated with said first and second target fusion transcripts but not with said third target fusion transcript;
said first reaction being carried out under one-step real time RT-PCR reaction conditions suitable for nucleic acid amplification and hybridization of complementary nucleic acid sequences, wherein an amplification product is formed if the sample contains said common target nucleic acid sequence; and,
c) detecting an amplification product specifically associated with at least one of said fusion transcripts by detecting said probes.

18. The method of claim 17 wherein the presence an amplification product specifically associated with said third target fusion transcript has a different clinical significance than the presence of an amplification product associated with said first or said second target fusion transcript.

19. The method of claim 17 further including the step of
contacting a second portion of sample nucleic acid from step (a) in a second reaction with a pair of primers and probe specific for a third target nucleic acid sequence under one-step real time RT-PCR reaction conditions suitable for nucleic acid amplification and hybridization of complementary nucleic acid sequences, wherein said nucleic acid sequence is associated specifically with a fourth target fusion transcript and wherein an amplification product is formed if the sample contains said third target nucleic acid; and,
determining whether an amplification product specifically associated with at least one of said fusion transcripts is present as a result of said first reaction or said second reaction by detecting and examining said probes.

20. The method of claim 19 wherein said first reaction and said second reaction are carried out simultaneously.

21. A method of monitoring the progress and adequacy of treatment in a subject who has received treatment for APL which comprises:
a) contact said sample with a pair of oligonucleotide primers, said primer pair capable of amplifying a plurality of target translocation sequences, said target translocation sequences having common nucleic acid sequences flanking a heterogeneous breakpoint region;
b) further contact said sample and said primer pair from a) with a pair of probes comprising an upstream probe and a downstream probe, whereas the upstream probe comprising a nucleotide sequence that hybridizes to an amplified upstream target nucleic acid sequence resulting from the use of said primer pair in a polymerase chain reaction, said upstream target nucleic acid sequence located in the same exon as a critical breakpoint region in said heterogeneous breakpoint region; and the downstream probe comprising a nucleotide sequence that hybridizes to an amplified critical target nucleic acid resulting from the use of said primer pair in said polymerase chain reaction, said critical target nucleic located in a critical breakpoint region, said critical breakpoint region not present in at least one of said plurality of translocation sequences;
c) amplify the sample nucleic acid sequence by conducting an one-step real time RT-PCR reaction under the conditions suitable for nucleic acid amplification and hybridization of complementary nucleic acid sequences.

22. A kit for use in detecting the presence of a breakpoint 5′ to (nt) 1709 of a t(15;17) PML/RARα gene translocation in a sample suspected of containing said translocation comprising:
(a) a container;
(b) a primer pair of claim 7 in said container
(c) A first probe comprising the nucleotide sequence 5′ AGG AAG TGC AGC CAG ACC CAG TGC-3′ (SEQ. ID NO.2) that hybridizes to an amplified target nucleic acid sequence resulting from the use of the primer pair of claim 1 in a polymerase chain reaction said target nucleic acid sequence common to bcr1, bcr2 with breakpoint 3′ to (nt) 1709 and bcr2 with breakpoint 5′ to (nt) 1709.
(d) A second probe comprising the nucleotide sequence 5′ AAG TGA GGT CTT CCT GCC CAA CAG CA-3′ (SEQ. ID NO.3) that hybridizes to an amplified target nucleic acid sequence resulting from the use of the primer pair of claim 1 in a polymerase chain reaction, said target nucleic acid sequence common to bcr1 and bcr2 with breakpoint 3′ to (nt) 1709 and located at a critical breakpoint region 3′ to said second probe and immediately 5′ to (nt) 1709.

23. A method of detecting the presence of target nucleic acid translocation sequences and further detecting heterogeneous breakpoints in said translocation sequences in a sample suspected of containing said translocation, comprising:
a) contact said sample with a pair of oligonucleotide primers, said primer pair capable of amplifying a plurality of target translocation sequences, said target translocation sequences having common nucleic acid sequences flanking a heterogeneous breakpoint region;
b) further contact said sample and said primer pair from a) with a first and second probe comprising an upstream probe and a downstream probe, wherein the upstream probe comprises a nucleotide sequence that hybridizes to an amplified upstream target nucleic acid sequence resulting from the use of said primer pair in a polymerase chain reaction, said upstream target nucleic acid sequence located in the same exon as a critical breakpoint region in said heterogeneous breakpoint region; and wherein the downstream probe comprises a nucleotide sequence that hybridizes to an amplified critical target nucleic acid resulting from the use of said primer pair in said polymerase chain reaction, said critical target nucleic located in a heterogenous breakpoint region, said critical target nucleic not present in at least one of said plurality of translocation sequences;
c) amplify the sample nucleic acid sequence by conducting an one-step real time RT-PCR reaction under the conditions suitable for nucleic acid amplification and hybridization of complementary nucleic acid sequence

24. The method of claim 23 further comprising
Further contacting said sample at step b) with a third probe comprising a further downstream probe, said further downstream probe comprising a nucleotide sequence that hybridizes to an amplified further downstream target nucleic acid sequence resulting from the use of the primer pair in a polymerase chain reaction, said further downstream target nucleic acid sequence present in only one of said plurality of translocation sequences

25. A probe comprising the nucleotide sequence 5′ ACG TGG CCA GTG GCG CCG-3′ (SEQ. ID NO.10 ) that hybridizes to an amplified target nucleic acid sequence resulting from the use of the primer pair of claim 7 in a polymerase chain reaction, said target nucleic acid sequence present in bcr1 and not present in bcr2.


SEQUENCE LISTING (10 sequences)

SEQ ID NO: 1 - DNA, 21 bp, Artificial Sequence
  bcr1 and bcr2 foward primer
tctccaatacaacgacagccc

SEQ ID NO: 2 - DNA, 24 bp, Artificial Sequence
  bcr1 probe with 5′ labeled with 6-FAM
aggaagtgcagccagacccagtgc

SEQ ID NO: 3 - DNA, 26 bp, Artificial Sequence
  bcr2 probe with 5′ labeled with VIC
aagtgaggtcttcctgcccaacagca

SEQ ID NO: 4 - DNA, 21 bp, Artificial Sequence
  bcr3 forward primer
gatggcttcgacgagttcaag

SEQ ID NO: 5 - DNA, 26 bp, Artificial Sequence
  bcr3 probe with 5′ labeled with 6-FAM
aggacctcagctcttgcatcacccag

SEQ ID NO: 6 - DNA, 24 bp, Artificial Sequence
  bcr1, bcr2, bcr3 reverse primer
gcactatctcttcagaactgctgc

SEQ ID NO: 7 - DNA, 25 bp, Artificial Sequence
  beta-actin forward primer
tcacccacactgtgcccatctacga

SEQ ID NO: 8 - DNA, 25 bp, Artificial Sequence
  beta-actin reverse primer
cagcggaaccgctcattgccaatgg

SEQ ID NO: 9 - DNA, 26 bp, Artificial
  beta-actin probe with 5′ labeled with VIC
atgccctcccccatgccatcctgcgt

SEQ ID NO: 10 - DNA, 18 bp, Artificial Sequence
  bcr2 long probe with 5′ labeled with VIC
acgtggccagtggcgccg